(19) United States
(12) Patent Application Publication
(10) Pub. No.: US 2023/0226136 A1
(43) Pub. Date: Jul. 20, 2023

(54) A SYNERGISTIC FORMULATION FOR MANAGEMENT OF RESPIRATORY PATHOGENS INCLUDING CORONAVIRUSES

(71) Applicant: Shefali SABHARANJAK, Bangalore (IN)

(72) Inventor: Shefali SABHARANJAK, Bangalore (IN)

(21) Appl. No.: 17/777,159

(22) PCT Filed: May 6, 2021
(86) PCT No.: PCT/IB2021/053825

§ 371 Date: May 16, 2022

(30) Foreign Application Priority Data
May 7, 2020 (IN) 202041019397
Mar. 5, 2021 (IN) 202141009275

(51) Int. Cl. A61K 38/08; A61K 38/06; A61K 38/05; A61K 31/714; A61K 47/18; A61P 31/14

(52) U.S. Cl. CPC A61K 38/08; A61K 38/06; A61K 38/05; A61K 31/714; A61K 47/186; A61P 31/14

(57) ABSTRACT

A synergistic non-toxic formulation for management of respiratory pathogens including coronaviruses and the process of preparing the synergistic formulation. The effective, non-toxic composition comprises of biological peptides including calcium chelating peptide and zinc chelating peptides, protease inhibitor such as 9-Arginine-peptide and inhibitor of viral replication enzyme such as vitamin B12 to reduce the presence of respiratory viruses including coronaviruses. The formulation is a cocktail of ingredients having a synergistic effect which is capable of being administered through various means including by way of inhalation, as nasal spray or an oral pump or via nebulizer to inhibit the entry and replication of respiratory pathogens. The combination of the formulation is such that it can be customized for a number of viral infections. The formulation is economical, self-administrable and effective even in low doses.

This complete specification is a cognate of provisional appl. no 202041019397 titled “A formulation of ion chelator and protease inhibitor to reduce respiratory viral infection” dated 7 May 2020 and provisional appl. no. 202141009275 titled “A synergistic formulation for preventive management of respiratory pathogens including coronaviruses” dated 5 Mar. 2021

### FIELD OF THE INVENTION

The present invention relates to a synergistic formulation for management of respiratory pathogens including coronaviruses and the process of preparing the synergistic formulation. More specifically it discloses a non-toxic composition of ion chelator, protease inhibitor and inhibitor of viral replication enzyme to reduce the presence of respiratory viruses including coronaviruses. The formulation is a cocktail of ingredients having a synergistic effect which is capable of being administered through various means including by way of inhalation, as nasal spray or an oral pump or via nebulizer.

### BACKGROUND OF THE INVENTION

Virus is a submicroscopic infectious agent that replicates only inside the living cells of an organism. Viruses can infect all types of life forms, from animals and plants to microorganisms, including bacteria resulting in varied symptoms based on the severity of the infection.

There are a large number of microscopic and submicroscopic agents including bacteria and viruses that may cause infections on inhalation. These causative agents of disease in human are capable of having significant impact on morbidity and mortality. The specific form of viruses includes respiratory viruses, which are the most frequent causative agents of disease in human causing significant morbidity and mortality. The studies show that eight human respiratory viruses circulate commonly in all age groups and are recognized as adapted to efficient person-to-person transmission. The respiratory viruses such as Respiratory Syncytial Virus (RSV) and influenza are well known to infect the lower airway, and both can cause bronchitis, bronchiolitis and pneumonia. RSV has traditionally been considered to be an upper airway pathogen because of its association with common cold symptoms and the observation that the viral replicates in optimum level at 33° C.-35° C., which approximates to temperatures in the upper airway.

SARS coronavirus (SARS-CoV) and avian influenza virus H5N1 have emerged in recent years as threat to public health. SARS-CoV has been out of circulation since 2003 whereas the avian influenza virus H5N1 has caused limited outbreaks of human infection.

Viral infections commonly affect the upper or lower respiratory tract. Although respiratory infections are classified by the causative virus, for example influenza, they are generally classified clinically according to syndrome for example the common cold, bronchiolitis, croup, pneumonia. Although specific pathogens commonly cause characteristic clinical manifestations, each can cause many of the viral respiratory syndromes.

Further, the severity of viral respiratory illness varies widely and is more evident in older patients and infants. Morbidity may result directly from viral infection or may be indirect due to exacerbation of underlying cardiopulmonary conditions or bacterial superinfection of the lung, paranasal sinuses or middle ear. Bacteria such as Mycobacterium tuberculosis infect the lungs of the host leading to the formation of granulomas in which the bacteria multiply, and are spread through the respiratory tract of the host, in the form of droplets expelled through coughing or sneezing.

Viral respiratory infections are clinically diagnosed based on symptoms and local epidemiology. Viral respiratory infections are spread when an infected person talks, coughs or sneezes, thus releasing small droplets containing infectious agents into the air. The droplets in the air may be inhaled by other people in nearby vicinity. The viruses are also spread by direct contact with a sick person or indirect contact with hands, tissues or other articles.

The outbreak of a new form of coronavirus in 2020 has affected most of the countries in the world. Coronaviruses are a group of enveloped Ribonucleic acid (RNA) viruses with the largest RNA genome among all the known viruses. COVID-19, a disease caused by a novel Severe Acute Respiratory Syndrome Coronavirus 2 (SARS-CoV-2) virus results in similar symptoms as Severe Acute Respiratory Syndrome (SARS). The SARS-CoV-2 pandemic has shown that the emergence of new viruses, especially respiratory and airborne viruses is a new health threat. The viral outbreaks of SARS-CoV-2 have stirred panic and emergency on public health around the world and the number of infections continues to rise. However, cause and consequence of the associated pneumonia remain unknown.

The most common symptoms of coronavirus are fever, cough, shortness of breath and breathing difficulties. In more severe cases of infection, it can also cause pneumonia, severe acute respiratory syndrome (SARS), severe acute respiratory illness (SARI) and even death. The period within which the symptoms would appear is 2-14 days.

The outbreak of viral infection can be controlled as preventive or treatment measures. However, there is no drug or composition, which is effective in alleviating the symptoms or to reduce the severity of the infection caused by coronavirus in humans.

The progression of viral infections is related to immunity of the person. Therefore, many researchers are focusing on improvement of the immune system, which may help in defending the pathogens. Vitamins are known to boost the immune system. Vitamins are either water soluble of fat soluble nutrients essential to the health and functioning of the immune system. Vitamins enhance the pathogen fighting effects of monocytes, macrophages and white blood cells that are important components of the immune defense, thus decreasing inflammation, which helps to promote immune response. It is observed that low vitamin levels are associated with an increased risk of upper respiratory tract infections and studies have shown that supplementation of vitamins improve immune response and protect against respiratory tract infections. It is also noted that vitamin supplementation improves response to antiviral treatments in people with certain infections.

The efficacy of a drug depends on the mode of administration of the formulation or composition. A combination of different antiviral agents that may inhibit the viral replication may be effective in treatment of respiratory viral infections. However, the existing antiviral compositions are administered through oral delivery in the form of pills, which may require more time to reach the site of action i.e., lung tissue, and is required to travel through blood stream or digestive tract. Further, as most of the antiviral drugs need to be processed by the liver, for the drug to be eliminated through feces and urine, the composition should be safe without inducing any adverse effects.

The available compositions may not be effective in reducing the viral infection and a formulation targeting the replication of virus or an effective mode of administration is not available. Hence, there is a need for a formulation, which is effective against viral respiratory diseases including coronavirus.

The outbreak of infection can be controlled as preventive or curative measures. For prevention of infection, strategies to block the entry of viruses within the respiratory system and preventing them from gaining access to the bloodstream are most effective.

The Patent Application No. US20170158697A1 entitled “Protein Kinase Inhibitors” discloses a family of protein kinase inhibitors, more specifically directed to inhibitors of the members of the Tec or Src protein kinase families. The invention also relates to processes for the preparation of these compounds, the pharmaceutical composition comprising them, and their use in the treatment of proliferative, inflammatory, infectious or autoimmune diseases, disorder or condition in which protein kinase activity is implicated.

The Patent Application No. US20170158697A1 entitled “Methods for treating herpes virus infections” discloses methods for treating, killing, and/or inhibiting the growth of Herpes viruses in human subjects comprising topically administering to a human subject in need thereof, a nano emulsion composition having antiviral properties.

The Patent Application No. WO2019046664A1 entitled “Compositions and methods for protecting against pathogens and irritants” discloses methods and compositions for enhancing the ability of the respiratory membranes to filter airborne pathogens and protect a subject from respiratory infections that result from inhalation or ingestion of such pathogens. In particular, the disclosure provides antimicrobial compositions that prevent and treat respiratory infections caused by bacteria, fungi, and viruses.

The Patent Application No. US20090209640A1 entitled “Prevention of and countermeasures against viral infection” relates to a method for treating viral infection, which includes administering arginine and a high dose of vitamin C. It also relates to an agent for treating viral infection, which includes arginine and coated vitamin C.

The available compositions of the aforementioned applications may not be effective in reducing the infection since they disclose neither any formulation targeting the replication of virus nor an effective mode of administration. Moreover, they do not disclose any drug or composition which is effective in alleviating the symptoms or to reduce the severity of the infection caused by coronavirus in humans.

One of the main drawbacks of the existing state of art is that none of them disclose a formulation that is capable of being prophylactic against a variety of respiratory pathogens especially viral pathogens. Yet another drawback is that the existing compositions are administered through oral delivery in the form of pills, which may require more time to reach the site of action i.e., lung tissue and is required to travel through blood stream or digestive tract. Further, as most of the antiviral drugs need to be processed by the liver, for the drug to be eliminated through feces and urine, the composition should be safe without inducing any adverse effects. All the previously reported compositions have to be ingested and show their activity after passing through the blood stream, which increases their delivery time to the site of action. There is no reported formulation that can be administered through inhalation, either as nasal spray or an oral pump or via nebulizer and which is non-toxic to human cells. Moreover, currently, there are no inhalable solutions that can stop infections by respiratory pathogens-viruses especially SARS-CoV-2, bacteria or mycobacteria.

### OBJECT OF THE INVENTION

The object of the present invention is to provide a synergistic formulation for effective management of respiratory pathogens including coronaviruses such as SARS-CoV-2, and bacteria such as mycobacteria.

Another object of the present invention is to provide a synergistic formulation that is capable of being administered in multiple ways including by way of inhalation through nasal spray or oral pump or via nebulizer, for the prevention of respiratory infections.

Yet another object of the present invention is provide a synergistic formulation having a molecular composition which is non-toxic to human cells.

### SUMMARY OF THE INVENTION

The invention discloses a synergistic formulation for effective management of respiratory pathogens including viruses particularly SARS-CoV-2 and other coronaviruses. The synergistic formulation comprises effective combination of ion chelator, protease inhibitor(s), inhibitors of action of pathogenic protein and lipid factors and inhibitor of replication enzyme(s) of pathogen. The formulation is a non-toxic synergistic cocktail of ingredients, which is capable of being administered in multiple ways including by way of nasal spray or oral pump or via nebulizer and is therefore effective in inhibiting the entry and replication of respiratory pathogens.

The invention discloses a specific combination of ion chelators such as calcium chelating peptide and zinc chelating peptides, protease inhibitor such as 9-Arginine-peptide and inhibitor of viral replication enzyme such as vitamin B₁₂.

The formulation of the present invention includes a combination of biological peptides which are non-toxic to human cells especially the lung cells. The formulation is effective in preventing the onset of viral infections and reduces the infective events even upon exposure to the virus. The combination of the formulation is such that it can be customized for a number of viral infections. The components of the formulation act synergistically to prevent the attachment of virus to the host cells and inhibit viral replication and viral protein priming, thus, reducing the chances of infections.

The ion chelators used in the formulation of the present invention chelate the calcium and zinc ions, thereby inhibiting viral proteins and preventing inflammation of the cells. The formulation of the present invention is biodegradable and non-toxic. The formulation has been shown in in vitro experiments to reduce up to 90% viral loads. It is an effective and economic formulation which can be safely administered against a variety of respiratory pathogens especially viral pathogens including coronavirus.

Accordingly, the present invention discloses a synergistic formulation for management of respiratory pathogens including coronaviruses. This formulation comprises of a combination of at least one ion chelator, at least one protease inhibitor and at least one inhibitor of viral replication enzyme. The formulation has been found to be efficacious and non-toxic to human cells.

The ion chelator(s) in the synergistic formulation of the present invention are selected from calcium chelating peptides or zinc chelating peptides. Indeed, both can be used in the composition. The calcium chelating peptides are selected from Phenylalanine-Aspartic acid-Histidine-Isoleucine-Valine-Tyrosine (FDHIVY), in a concentration ranging from 0.1 g/mL to 2.0 g/mL. The zinc chelating peptides are selected from Asn-Cys-Ser, Ser-Met dipeptide and Leu-Ala-Asn tripeptide in a concentration ranging from 0.1 g/mL to 2.0 g/mL.

The protease inhibitor included in the formulation of the present invention is preferably 9-L-Arginine-peptide at a concentration ranging from 5 uM to 400 uM.

The inhibitor of viral replication enzyme in the formulation of the present invention are vitamin B, vitamin D or more specifically vitamin B₁₂. The concentration of vitamin B₁₂ is in the range of 0.5 mg/mL to 5 mg/mL, preferably 100 mg/ml. In addition, the formulation also comprises a preservative at a concentration 0.01% w/v.

The present invention also discloses the process of preparing a synergistic formulation for the management of respiratory pathogens including coronaviruses, said process formulation comprising of synthesizing the peptides, purifying to >98% purity and storing the said peptides in dry state. The said peptides are weighed in quantities corresponding to 100× to 1000× concentration of the final concentration. The said peptides are formulated into a combination of specific concentration of each peptide. Vitamin B₁₂ is added to the said specific formulation of peptides in one tube and stored as a dry formulation. This is the synergistic combination of peptides to be used as a nasal-oral formulation. At the time of use of the formulation, the stored dry peptide combination is dissolved in phosphate-buffered saline, to be used for administration either as nasal spray or via oral pump or through a nebulizer.

More specifically the present formulation includes the efficacious synergistic formulation comprising 0.1 g/mL to 2.0 g/mL of calcium and zinc chelating peptide, 5 uM to 400 uM of 9-L-Arginine-peptide, and Vitamin B₁₂ in the range of 0.5 mg/mL to 5 mg/mL, and a preservative benzalkonium chloride at a concentration of 0.01% w/v.

The efficacy of the formulation of the present invention, also referred to as EvoBO2020, has been tested out in the following two combinations of peptides:
(a) In one embodiment, the formulation comprises of 9-Arg peptide at a concentration in the range of 5 uM to 400 uM, in combination with FDHIVY peptide at a concentration in the range of 0.1 g/ml to 2.0 g/ml and Vitamin B₁₂ at a concentration in the range of 0.5 mg/ml to 5 mg/ml.
   More specifically, the formulation in one embodiment comprises of 9-Arg peptide at a concentration in the range of 5 uM to 400 uM, more preferably 40 uM, in combination with FDHIVY peptide at a concentration in the range of 0.1 g/ml to 2.0 g/ml, more preferably 0.9 g/ml and Vitamin B₁₂ at a concentration in the range of 0.5 mg/ml to 5.0 mg/ml to, more preferably 100 mg/ml.
(b) In another embodiment, the formulation comprises of 9-Arg peptide at a concentration in the range of 5 uM to 400 uM, in combination with FDHIVY peptide at a concentration in the range of 0.1 g/ml to 2.0 g/ml and A1-AT-RC1 peptide at a concentration in the range of 5 uM to 200 uM.
   More specifically, the formulation in this embodiment comprises of 9-Arg peptide at a concentration in the range of 5 uM to 400 uM, more preferably 40 uM, in combination with FDHIVY peptide at a concentration in the range of 0.1 g/ml to 2.0 g/ml to, more preferably 0.9 g/ml and A1-AT-RC1 peptide at a concentration in the range of 5 uM to 200 uM to, more preferably 20 uM.

The formulation in both embodiments above have been prepared using synthetic peptides. Peptides like Zinc-chelating peptides, poly-Lysine peptides and Anti-HAT peptides can be possible addition to the formulation to increase their scope against other viruses and for use of this combination of peptides as an adjuvant in lung cancer and for preparation of inhalable vaccines.

The synergistic formulation of the present invention is capable of being administered in multiple ways including by way of inhalation through nasal spray or oral pump or via nebulizer, for the prevention of respiratory infections.

### BRIEF DESCRIPTION OF THE DRAWINGS

The foregoing and other features of embodiments will become more apparent from the following detailed description of embodiments, when read in conjunction with the accompanying drawings.

FIG. 1(a): Determination of IC-50 value of Positive Control (Remdesivir)

FIG. 1(b): Determination of IC-50 value of the formulation of the present invention referred to as EvoBO2020

FIG. 2: Depicts the anti-viral effect of formulation of the present invention referred to as EvoBO2020 in the plaque assay

FIG. 3: Depicts the plaque assay data for the positive control, Remdesivir.

FIG. 4: Depicts the plaque assay data for the formulation of the present invention referred to as EvoBO2020.

FIG. 5: Depicts the bar graph of absorbance 540 nm in the MTT Quantitation assay for lung cells exposed to EvoBO2020 formulation

### DETAILED DESCRIPTION OF THE OF THE PREFERRED EMBODIMENT

In order to more clearly and concisely describe and point out the subject matter of the claimed invention, the following definitions are provided for specific terms, which are used in the following written description.

The term “Protease” refers to an enzyme that catalyzes proteolysis, the breakdown of proteins into smaller polypeptides or single amino acids. The term “Viral Replication” refers to a process of formation of biological viruses during the infection in the target host cell. The term “Furin” refers to an enzyme responsible for the proteolytic cleavage of viral envelope polyprotein precursor prior to viral infections into human airway cells. Furin is expressed in cells that line the human airways and in lung tissues, both extracellularly and in intracellular compartments.

The term “Neuropilin-1” refers to are transmembrane glycoproteins that serve as cell surface receptors for various ligands involved in angiogenesis. The term “ion chelator” refers to small molecules that bind very tightly to metal ions. The term “Polymerase” refers to an enzyme that synthesizes long chains of polymers or nucleic acids.

Generally, viruses are not capable of self-replication and require a living host cell for replication and multiplication. The viruses exploit host cell mechanisms to survive. Proteases plays an important role in the binding of virus to human cells, leading to endocytic uptake of the viruses and replication of viruses. The viral RNA is translated into a polypeptide sequence, which is assembled in a long chain that includes several individual proteins such as reverse transcriptase, protease, integrase etc. These enzymes must be cleaved from the longer polypeptide chain before they become functional. Viral protease cleaves the long chain into its individual enzyme components that facilitate the production of new viruses. Hence, viral proteases play an important role in replication of viral genome and multiplication of virus.

Furin is a human protease involved in proteolytic cleavage of viral envelope polyprotein and associated with the development of infection. The use of Furin protease inhibitor 9-L-Arginine-peptide inhibits the ability of the protease to cleave the viral S-glycoprotein and entry of the virus into lungs thus interfering with continued infection. The inhibition of Furin also interferes with the processing of viral proteins and packaging into new virus particles within infected cells.

The entry of SARS-CoV-2 in cells depend on binding of the spike (S) coat protein to angiotensin-converting enzyme2 (ACE2), which is present on the plasma membrane of airway epithelial cells, goblet secretory cells, and type II pneumocytes, and on S protein priming by the serine protease transmembrane serine protease 2 (TMPRSS2). In the coronavirus S protein priming, the transmembrane serine protease 2 (TMPRSS2) is regarded as one of the main priming proteases, which is an important step for binding of the S protein to the angiotensin-converting enzyme 2 (ACE2) receptor before cell entry. Therefore, targeting the host extracellular proteases, such as TMPRSS2, and other proteases has several advantages over targeting viral proteins. Serine residues have been found to be important catalytic residues present in all extracellular transmembrane anchored proteases expressed in lung cells.

The protease inhibitors of the present formulation are effective in inhibiting the binding of respiratory viruses to the airway epithelial tissues in the respiratory pathway which, in case of an infection process, is facilitated through the action of proteases. The specific peptides of the formulation also prevent the endocytosis of viruses especially SARS-CoV-2 which is enabled by host protein mediated cleavage of viral proteins.

The formulation of the present invention provides for a poly-lysine peptide of 7-10 residue length to temporarily inactivate extracellular proteases by binding to their catalytic sites. The poly-lysine peptide is preferably of molecular size of 0.1-1 nm. A possible longer or shorter version of this peptide may also be included in the formulation,

It is significant to note that other extracellular proteases expressed in lung cells include a class of proteins called Meprin proteases. The inclusion of zinc chelating peptides like: Asn-Cys-Ser tripeptide, Ser-Met dipeptide, and Leu-Ala-Asn tripeptide temporarily inhibits the activities of the Meprin class proteases, for as long as the formulation is present on the extracellular surface of lungs.

Neuropilin-1 (NRP-1) is a cell-surface receptor that plays an essential role in angiogenesis, regulation of vascular permeability, and the development of the nervous system. VEGF-A165 and other ligands of NRP-1 possess a C-terminal CendR sequence that interacts with the b1 domain of NRP-1 and causes cellular internalization and vascular leakage. CendR peptides bind to neuropilin-1 (NRP-1) on the target cells.

Neuropilin-1 is a co-receptor for SARS-CoV-2-highly expressed in lungs. Neuropilin binds to R-XX-R peptide motifs at the C-terminal end using its VEGF binding domain. R-X-XR- is produced by Furin cleavage of SARS-CoV-2 S-glycoprotein cleavage and resultant S-peptide. This CendR (C end Rule -R-X-X-r/K at the C-terminus) pathway stimulated transcytosis of extracellular large molecules independent of known endocytic paths (clathrin/wortmannin and other pinocytic inhibitors). The intracellular signaling/binding of other proteins to NRP-1 are prevented by the components of the present invention. Also, prevention of formation of the R-XX-R peptide fragment from the S-glycoprotein of SARS-CoV-2 by the action of proteases is achieved by the protease inhibitors used in the formulation of the present invention. The inactivation of such extracellular proteases is relieved only when the peptide and protease complex is degraded by natural cellular processes.

Compounds that inhibit viral replication target the enzymes used in viral replication. Virus replicates inside the host cell by manipulating the host enzymes to make multiple copies of itself. The use of viral replication inhibitor plays an important role in the efficacy of the formulation. The composition of the present invention also comprises of inhibitors of viral replication which play an important role in the efficacy of the formulation. The replication inhibitors of the present formulation, Vitamin B₁₂ can stop the steps in the infection process of respiratory pathogens by preventing the multiplication of viruses facilitated by the activity of the enzyme RNA-dependent RNA polymerase, a key enzyme in viral replication. The inhibition of RNA-dependent RNA polymerase reduces both the production of new viruses as well as lateral spread of the SARS-CoV-2 virus from cell to cell.

The formulation of the present invention is a synergistic formulation for management of respiratory pathogens including coronaviruses. The formulation of the present invention comprises of a mixture of specific combination of biological, non-toxic peptide molecules, designed to be administered in multiple ways including by way of inhalation through nasal spray or oral pump or via nebulizer, for the prevention of respiratory infections.

In one embodiment, this synergistic formulation comprises of ion chelator selected from zinc chelating peptide and calcium chelating peptides. These ion chelators inhibit the activities of proteases for as long as the formulation is present on the extracellular surface of lungs. This inhibition is likely to prevent the priming of pathogenic proteins by the proteases.

Calcium, a secondary metabolite plays an important role in signaling of calcium channels or calcium pumps. The use of ion chelator i.e. calcium chelating peptide selected from FDHIVY and other similar calcium-chelating peptides in the formulation helps in chelating calcium, thereby inhibiting activity of human inflammatory protein called C Reactive Protein (CRP) and SARS-CoV-2 protein E, which is a calcium channel protein required to maintain infectivity of SARS-CoV-2 virus.

Neuropilin also has 2 calcium binding motifs at positions 195, 209 250. Cytoplasmic tails of neuropilin-sequence S-E-A-COOH binds to NIP which in turn mediates Clathrin mediated endocytosis. Calcium bound to these residues are further chelated by the addition of calcium chelating peptides-FDHIVY and other similar calcium-chelating peptides. Hence, the addition of a peptide with the sequence -Serine-Glutamic Acid-Alanine- (-S-E-A-) is able to stop the endocytosis of the SARS-CoV-2 virus that is enabled by the Furin-dependent cleavage of the S-glycoprotein.

The synergistic formulation of the present invention has been tested for antiviral activity on SARS-CoV-2 and for its non-toxicity on lung cells. Positive Control used in the experiments is Remdesivir. The formulation of the present invention is referred to as Test Material also referred to as EvoBO2020.

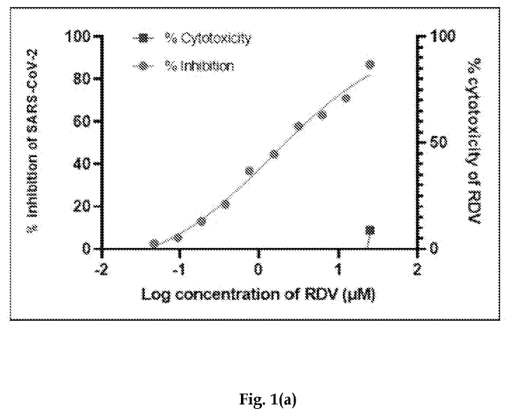

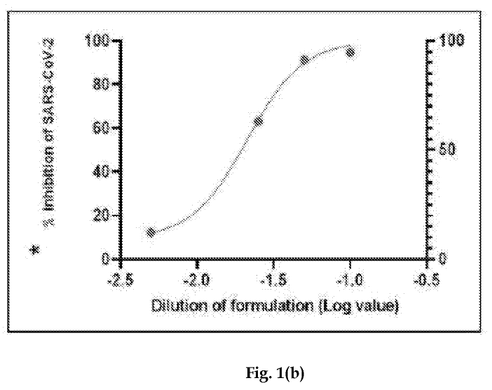

FIG. 1(a) depicts the results of IC-50 plaque assay against SARS-CoV-2, with the positive control Remdesivir, which is a known antiviral drug. The IC-50 value of Remdesivir was calculated by considering the top and baseline value 100 and 0 respectively. As is depicted in the figure, the percentage cytotoxicity of Remdesivir increased with the increase in its concentration. The IC-50 reported for Remdesivir was 2.14 μM, which is acceptable based on internal and literature data.

FIG. 1(b) depicts the results of IC-50 plaque assay against SARS-CoV-2, with the formulation of the present invention also referred to as EvoBO2020 in test reports. The IC-50 of the test material was calculated by considering the top and baseline value 100 and 0 respectively. The absolute IC-50 was calculated to be 0.02 dilutions against SARS-CoV-2.

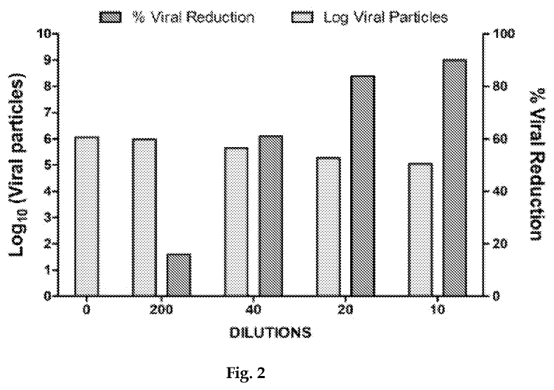

FIG. 2 depicts the anti viral effect of the formulation of the present invention also referred to as EvoBO2020 in test reports. The formulation of the present invention was tested for its efficacy in reducing the SARS-CoV-2 infectivity. 1:200, 1:40, 1:20 and 1:10 dilutions of the test formulation were tested against the virus.

The green bars in FIG. 2 depict log viral particles and the blue bars depict the percentage reduction of viral particles by different dilutions of the test material. Viral RNA was extracted by MagMAX™ Viral/Pathogen Extraction Kit Fosun COVID-19 RT-PCR Detection Kit.

As can be inferred from the FIG. 2, with increase in concentration of the formulation of the present invention, the viral load decreases. From the FIG. 2, it is clear that the 1:10 dilution of the test material drastically reduces the viral load up to 90%.

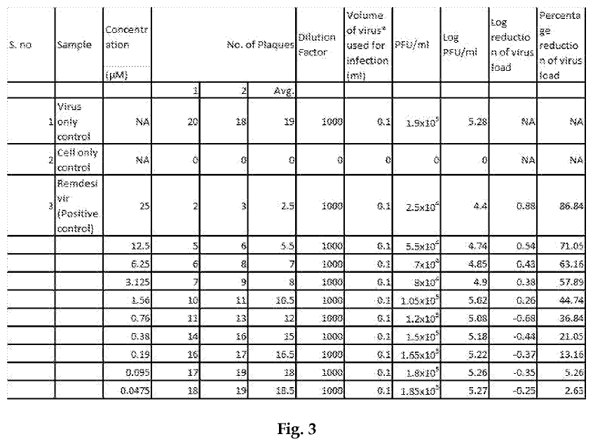

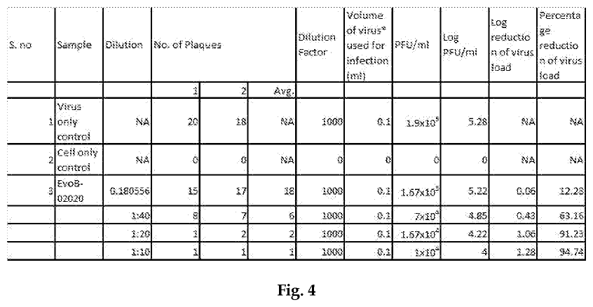

The same result can be explained with reference to FIGS. 3 and 4. The table in FIG. 3 depicts the plaque assay data for the positive control, Remdesivir. The highest concentration of Remdesivir tested (25 μM) caused reduction in viral load up to 86.84%. This is in contrast to the results of the plaque assay conducted with EvoBO2020, as disclosed in FIG. 4, wherein, the highest concentration of EvoBO2020 tested (1:10 dilution) caused a drastic reduction in viral load up to 94.74%. Thus, it can be inferred from the viral plaque assay that the synergistic formulation of the present invention showed 15%, 61%, 84% and 90% viral reduction at 1:200, 1:40, 1:20 and 1:10 dilutions respectively. The viral particle number reduced from 10^{6.0} to 10^{5.0} at 1:10 dilution.
The regression equation for viral particles Vs Ct value of the N-gene specific to SARS-CoV2 virus (y=−4.9474x+39.723, R²=0.9964)
   (X=Number of viral particles, y=Ct value)
Number of viral particles are calculated using X=(39.723−Ct_{N}−gene_{@different conc.})/4.9474.
Percentage Viral reduction=N_{c}−Nₜ×100/N_{c}
   where, N_{c} denotes number of viral particles without drug (control) and Nₜ denotes number of viral particles with drug (test)

Therefore, it can be inferred that the synergistic formulation of the present invention reduces viral load of SARS-CoV-2 significantly.

The formulation of the present invention comprises of a combination of ion chelator, protease inhibitor and inhibitor of viral replication enzyme. The formulation comprises a non-toxic synergistic cocktail of ion chelator, protease inhibitor and inhibitor of viral replication enzyme at a specific concentration for management of infections caused by the respiratory viruses.

Table 1 depicts the concentration of the biological, non-toxic peptide molecules included in the formulation of the present invention. The synergistic formulation of the present invention is prepared in various combinations of the constituents including protease inhibitor, 9 Arg, ion chelator, FDHIVY, Poly-Lys (L8), Anti-HAT, and vitamin B₁₂. Each combination may include some of the above mentioned constituents depending on the use of the formulation. These concentrations of the components of the synergistic formulations of the present invention have been tested to be effective in 200 μL of the formulation.

TABLE 1
Effective concentrations of components of the formulation
of the present invention

| Actual concentrations | | | | |
|---|---|---|---|---|
| that were effective in | | | | |
| 200 uL | 1:30 dilution | | 1:20 dilution | |
| 9 Arg | 1.3 | mM | 2 | mM |
| A1-AT-RCL | 11.9 | μM | 17.85 | μM |
| FDHIVY | 0.16 | μg/ml | 0.25 | μg/ml |
| Poly-Lys(L8) | 1.3 | mM | 2 | mM |
| Anti-HAT | 13.33 | μM | 20 | μM |

Generally, benzalkonium chloride is used as a preservative. However, the formulation is not restricted to the use of benzalkonium chloride and any similar preservative can be used in the formulation. The formulation of the present invention is a combination therapy in the form of a solution capable of being administered either by way of inhalation through nasal spray or oral pump or via nebulizer.

The formulation administered orally or as nasal drops or nebulizers reduce the binding of the respiratory virus to the airway epithelial tissues. Such a formulation is non-toxic when used on cultured lung cells, is unlikely to cross the lung membrane and is biodegradable.

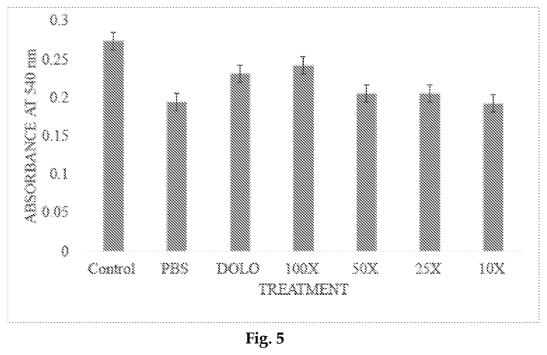

FIG. 5 depicts the results of MTT Assay-Quantitation-Experiment on 1-A549 lung cells when exposed to the formulation of the present invention, EvoBO2020. The cultured lung cells were exposed to PBS, DOLO, and different concentrations namely 100×, 50×, 25× and 10×, of the formulation of the present invention. Lung cell viability of was checked by addition MTT to all the test wells and reading absorbance at 540 nm. FIG. 5 denotes the absorbance of cells at 540 nm. The Absorbance values have been given in Table 1.

TABLE 2
Absorbance at 540 nm after addition of MTT

| Sample | Absorbance at 540 nm |
|---|---|
| Control 1 | 0.26 |
| Control 2 | 0.268 |
| Control 3 | 0.294 |
| PBS 1 | 0.189 |
| PBS 2 | 0.164 |
| PBS 3 | 0.229 |
| DOLO 1 | 0.218 |
| DOLO 2 | 0.237 |
| DOLO 3 | 0.239 |
| 100× 1 | 0.202 |
| 100× 2 | 0.27 |
| 100× 3 | 0.255 |
| 50× 1 | 0.174 |
| 50× 2 | 0.22 |
| 50× 3 | 0.223 |
| 25× 1 | 0.231 |
| 25× 2 | 0.188 |
| 25× 2 | 0.198 |
| 10× 1 | 0.193 |
| 10× 2 | 0.176 |
| 10× 3 | 0.208 |

The absorbance at 540 nm is linearly related to the cell activity. The average absorbances at 540 nm for control wells and subsequently treated wells with the test formulation are close. This indicates that the lung cells remain viable even after addition of 50-75 times the active concentration of the test formulation for extended periods (10-12 hours). Therefore, it is concluded that the test formulation of the present invention is non-toxic to the lung cells.

From the lung cell viability experiment it can be concluded that the formulation of the present invention can be delivered up to 50-75 times the active concentration without inducing toxicity.

The formulation of the present invention is safe and effective against activation of coronaviruses as well as other viruses due to combination of known biochemicals, which are integral part of the biochemical processes and hence does not induce any toxic effects. As seen in FIG. 5, the formulation has been shown to reduce infection with virus in in-vitro testing while at the same time being non-toxic to lung cells.

The ingredients of the formulation of the present invention are specifically selected to inhibit the activity of respiratory viruses especially coronavirus which, in turn, reduces the infection events. The Biomolecules used in the formulation have been synthesized and prepared to 98% purity.

The formulation of the present invention is prepared as solution, which can be administered in multiple ways including inhalation as nasal drops or through an oral pump or through nebulizer. This makes the formulation economically significant since it provides for effective and easy administration of the required dose directly into the site of action without requiring high dose of the drugs and is safe due to lower concentration of the drug. The administration of the formulation through inhalation is associated with effective and ease of administration or self-administration without the requirement of any specific skill or training for the medical care or health care staff.

This is a unique formulation that has been designed against SARS-CoV-2 but can be further developed to reduce respiratory infections significantly. This can be a significant addition to the personal protection strategy of people exposed to respiratory infections.

I claim:

1. A synergistic formulation for preventive management of respiratory pathogens including coronaviruses said formulation comprising
at least one ion chelator
at least one protease inhibitor and
at least one inhibitor of viral replication enzyme
in efficacious and non-toxic concentrations capable of being delivered orally, nasally and inhaled into lungs.

2. The synergistic formulation as claimed in claim 1, wherein, the said ion chelator(s) is selected from calcium chelating peptides and zinc chelating peptides.

3. The synergistic formulation as claimed in claim 2 wherein the calcium chelating peptides are selected from Phenylalanine-Aspartic acid-Histidine-Isoleucine-Valine-Tyrosine (FDHIVY).

4. The synergistic formulation as claimed in claim 3 wherein said calcium chelating peptides are at a concentration ranging from 0.1 g/mL to 2.0 g/mL.

5. The synergistic formulation as claimed in claim 2 wherein said zinc chelating peptides are selected from Asn-Cys-Ser, Ser-Met dipeptide and a Leu-Ala-Asn tripeptide at a concentration ranging from 0.9 g/mL to 1.1 g/mL.

6. The synergistic formulation as claimed in claim 1 wherein said protease inhibitor is preferably 9-L-Arginine-peptide at a concentration ranging from 5 uM to 400 uM.

7. The synergistic formulation as claimed in claim 1 wherein said inhibitor of viral replication enzyme is vitamin.

8. The synergistic formulation as claimed in claim 1 wherein said vitamin is selected from vitamin B.

9. The synergistic formulation as claimed in claim 1 wherein said vitamin B is preferably B₁₂

10. The synergistic formulation as claimed in claim 1 wherein said vitamin B₁₂ is at a concentration in the range between 0.5 mg/mL to 5.0 mg/mL

11. The synergistic formulation as claimed in claim 1 wherein said vitamin B₁₂ is preferably vitamin B₁₂ at a concentration in the range between 100 mg/mL to 1000 mg/mL.

12. The synergistic formulation as claimed in claim 1 wherein said formulation comprises a preservative at a concentration 0.01% w/v.

13. Process of preparing a synergistic formulation as claimed in claim 1 for preventive management of respiratory pathogens including coronaviruses said process comprising:
synthesizing said peptides and purifying to >98% purity,
storing said peptides in dry state,
weighing said stored peptides in quantities corresponding to 100× to 1000× of the final concentration,
combining said weighed peptides with vitamin B₁₂ in one tube and storing them as a dry formulation,
dissolving said dry formulation in phosphate-buffered saline at the time of use, for delivery of said synergistic formulation, orally, nasally and inhaled into lungs.

14. Synergistic formulation for preventive management of respiratory pathogens including coronaviruses said formulation comprising

| Ingredient | Concentration |
|---|---|
| Calcium chelating peptide | 0.1 g/ml to 2.0 g/ml |
| Zinc chelating peptide | 0.1 g/ml to 2.0 gm/ml |
| 9-L-Arginine-peptide | 5 uM to 400 uM |
| Vitamin B₁₂ | 0.5 mg/ml to 5 mg/ml |
| Alpha-A1-AT-RCL | 5 uM to 200 uM |
| benzalkonium chloride | 0.01% w/v |

15. The synergistic formulation as claimed in claim 1 wherein said formulation is capable of being delivered as a nasal spray, nebulizer or via personal oral pump.
